(19) United States
(12) Patent Application Publication
(10) Pub. No.: US 2015/0245547 A1
(43) Pub. Date: Aug. 27, 2015

(54) Method and use of organic and mineral admixtures for EMI and radioisotope shielding of air filtration media and building materials, for absorption of airborne particulates and for climate change mitigation

(71) Applicant: William L. Robinson, JR., Baltimore, MD (US)

(72) Inventor: William L. Robinson, JR., Baltimore, MD (US)

(21) Appl. No.: 13/999,406

(22) Filed: Feb. 24, 2014

(51) Int. Cl. H05K 9/00; C06B 49/00; B01J 21/06; B32B 37/24; B01J 20/16; B01D 53/04; G21F 9/02; B01D 53/62; A61K 33/14; A61L 9/015

(52) U.S. Cl. CPC H05K 9/0081; A61K 33/14; C06B 49/00; B01J 21/063; A61L 9/015; B01J 20/165; B01D 53/04; G21F 9/02; B01D 53/62; B32B 37/24; B01D 2255/50; B01D 2255/20707; B01D 2257/504; B01D 2258/0283; B32B 2037/243; B32B 2305/28; B32B 2307/724; B32B 2315/085; B32B 2315/18; B32B 2317/18; B32B 2607/02; B32B 2398/20

(57) ABSTRACT

The invention of high efficient multipurpose air filtration media for absorption of greenhouse gases such as CO₂ for climate change mitigation (lessening the severity of global warming) and absorption of airborne particulates (PM 0.5 μ-2.5 μ) and the electromagnetic and radioactive isotope shielding of building materials such as wall liners and wall coverings and reflective ceiling insulation using an absorbent organic admixture composed of a polysaccharide such as Hydroxypropylcellulose, a synergistic monosaccharide such as ethoxylated methylglucoside and de-ionized water and mineral additives such as TiO₂, Zeolites (Heu & Ca-A(5A)) and boehmites (Curie Formula) is disclosed.

### CLAIM OF BENEFIT OF EARLIER FILING DATES

This application claims benefit of the earlier filing dates, Feb. 16, 2010 and Jul. 7, 2011 of Non-Provisional application Ser. Nos. 12/545,741& 13/067,917 and 13/573,004 International Patent Nos. 1-2008-00779 (Vietnam National Patent) and 1-2011-144133 (Japan National Patent) in the name of the present Applicant, William L. Robinson, JR., of Baltimore, Md. and entitled “Method And Use Of Minerals Extracted From Fly Ash For EMI/RF/Microwave And X-Ray Shielding And Production Of Synthetic Diamonds and Thin Diamond Film Semiconductors and Diamond Wafers and Electrical Energy Storage Systems” and “Method And Use Of Organic And Mineral Admixtures For EMI And Radioisotope Shielding Of Building Materials Such As Glass Fiber Wall Liners, Reflective Ceiling Insulation And Electrically Conductive Or Resistive High Performance, High Strength Concrete”, respectively.

### BACKGROUND OF THE INVENTION

1. Field of the Invention

This invention relates to a method of increasing the tensile, flexural and compressive strengths and the EMI/RF/Microwave and radioactive isotope shielding of concrete, cement, gypsum or other pozzolan (alumina siliceous) such as fly ash using electroplated nickel oxide or copper coated stainless steel fibers, hydroxypropylcellulose and ethoxylated methylglucoside, petroleum coke powder or graphite and silica fume and non-radioactive natural zeolites such as Clinoptilolite and Phillipsite (as radioactive absorbing agents) and Titanium Dioxide (for microbial deactivation or decomposition).

This invention also relates to the use of zeolite based compounds to adsorb and absorb electromagnetic (RF) waves and airborne radioactive isotopes such as ¹³⁷Cs and ⁹⁰Sr and their subsequent use in the treatment of human cancers and for Absorption of greenhouse gases such as CO2 ozone and radon (and its progeny daughter product polonium).

This invention also relates to the use of using radiation absorbing wallpaper and radiation absorbing air filtration media and the combination of titanium dioxide for deactivation of airborne bacteria, viruses, mold and mildew and absorption of PM 0.5-2.5, etc.

2. Discussion of the Related Art

Cement is a widely used building material, but it lacks the ability to shield electromagnetic radiation. As the environment is increasingly sensitive to electronic pollution, the ability of a building to shield electromagnetic radiation is of increasing importance. Fly ash can be substituted for cement in concrete mixes for global construction of infrastructures saving energy, disposing of waste products, protecting the environment against global warming emissions, improving the quality of concrete and reducing cost. Ultra fine fly ash can be added to silica fume to enhance the strength of Concrete.

There has been a strong demand of late for high quality and light weight radioactive isotope shielded building materials such as wall coverings and ceiling insulations.

There has also been a demand of late for high efficient air filtration systems in areas of the world where accidental radiation exposure has become a reality, e.g. Fukushima, Japan.

3. Statement of Need

There is a need for protecting reinforcing steel adding to the longevity of concrete structures by preventing the penetration of waterborne contaminants and chloride-laden liquids that cause the corrosion of reinforcing steel.

There is a need for increased bonding strength and contact resistivity between cement and structural steel or steel fibers.

Because of the developments in electronics technology, there is a need for EMI/RF/Microwave Interference and radioactive particle shielding of air filtration media, building materials e.g. wall liners and concrete particularly in underground vaults containing power transformers and other electronics that are relevant to electric power and telecommunications and for deterring electromagnetic forms of spying.

There is a need for an environmentally friendly way to recycle ashes produced from the industrial combustion of coal and petroleum and the minerals and metals contained therein e.g. selenium, vanadium, nickel and holmium.

There is definitely a need for a way to trap and recycle radioactive nuclear fission products (isotopes) e.g. ¹³⁷Cs and ⁹⁰Sr and Ozone, Radon, NOx gases and CO₂ accidentally, intentionally or naturally released into the environment.

### GENERAL BACKGROUND

Electric utilities in the United States generate over 100 million tons of petroleum coke ash and coal fly ash as a by-product each year. Fly ash in particular is typically disposed of in landfills. Course fly ash ground to approximately 3.8 μm can produce high strength concrete and 25% cement replacement gave the highest compressive strength (100.3 MPa). A replacement of 25% cement will result in a 27% reduction in greenhouse gases produced from production of cement (680 Kg/ton of cement).

The cement industry is responsible for producing 5% of global CO2 emissions; 60% due to de-carbonization of non-renewable materials such as limestone and 40% due to heating cement kilns to 1500° C. using non-renewable fossil fuels.

Adding 0.90 vol. % stainless steel fibers (by weight) to cement improves strength by 23% equal to 2-3 times that of non-reinforced concrete. The dominant mechanisms of EM/RF/Microwave shielding for micron size (>100 nm) steel fibers is absorption. Nickel filaments of diameter 0.4 μm, as made by electroplating 0.1 μm diameter carbon filaments with nickel, have been shown to be particularly effective. They are known as nickel filaments because they are mostly nickel rather than carbon. A shielding effectiveness of 87 dB at 1 GHz has been attained in a polymer-matrix composite containing just 7 vol. % nickel filaments. Nickel is more attractive than copper, partly due to its superior oxidation resistance.

Shielding of 40 dB or more in the magnetic field ranging from 150 kHz to 16 MHz is needed for a 99% EMI block. This degree of shielding effectiveness is sufficient to for the construction of electromagnetic interference structures.

Binding Properties of Calcium Hydroxide or Hydrated Lime (CaCO₃) with HPC.

Calcium hydroxide or hydrated lime is the product of the hydration of lime and water:

Ca(OH)₂<========>CaO+H₂O

Lime is a soft, white amorphous powder with alkaline or slightly bitter taste. It has been shown that lime is solubilised in the presence of sugars and it has been observed in set Portland cements as hexagonal plate crystals (Lea, 1970). Lime reacts with carbon dioxide (CO₂) to form calcium carbonate (CaCO₃). This reaction which takes place in the presence of moisture is the cause of hardening of high calcium lime mortars.

Binding Properties of HPC with Steel Fiber and Cement

HPC and Ethoxylated methylglucoside (moisture barrier) binds together at the 1-3′ C-Terminal Domain. How does HPC bind to calcium in concrete? In the presence of water calcium located at the N-Terminal Cellulose Binding Domain in HPC will bind to calcium bonds at the 1-4′ β calcium bonding sites in cement.

The use of hydroxypropylcellulose or methylcellulose (0.4% to 0.8% by weight of cement) as an admixture in cement paste or concrete was found to increase the shear bond strength with steel reinforcing bar and steel fiber. The bond strength increased with increasing hydroxypropylcellulose or methylcellulose amounts. The contact electrical resistivity between cement and fiber or between concrete and reinforcing bar was not changed by addition of hydroxypropylcellulose or methylcellulose.

Trapping of Radioactive Fission Products (Isotopes) Using Non-Radioactive Stable Metallic Elements

Holmium (ho [symbol] mi [symbol] m/HOHL-mee- [symbol] m) is a chemical element with the symbol Ho and atomic number 67. Part of the lanthanide series, holmium is a relatively soft and malleable silvery-white metallic element, which is stable in dry air at room temperature. A rare earth metal, it is found in the minerals monazite and gadolinite. Holmium has the highest magnetic strength of any element and therefore is used for the pole pieces of the strongest static magnets. Because holmium strongly absorbs nuclear fission-bred neutrons, it is also used in nuclear control rods.

Zeolite chemistry is the distribution of silicon and aluminium atoms among the T sites. According to Lowensteins' rule, Al—O—Al linkages in zeolitic frameworks are forbidden. As a result, all aluminate tetrahedra must be linked to four silicate tetrahedra, and in general this is proved to be the case, but recent investigations into zeolites synthesized at high temperatures have shown non-Lowenstein distributions in sodalite materials. Aluminum ions are formed by losing 3 electrons making it neutrally charged. The combination of negatively charged silica and aluminum produces negatively charged ions that will absorb electromagnetic waves. Negative ions are a type of antioxidant present in nature that is reported to react with and break down toxins in the bloodstream. the range of SiO₄/AlO₄ ratios varies between zeolites. ZSM-5 is a high silicate zeolite, whereas zeolite X/Y can be prepared in high silicate forms, or high aluminate forms, but is usually produced with a Si/Al ratio close to unity with a fully ordered Si—Al distribution over the tetrahedral sites, in accordance with Lowenstein's rule.

The inclusion of aluminum into the zeolite structure has two major effects: an increase in the net negative charge—which are neutralized from protons hydrogen bonded to the lone pairs of the bridging oxygens. These acidic sites play a significant role in the zeolite catalytic activity. the materials become hydrophilic.

Zeolites are not only influenced by pH but also they are capable of affecting the solution pH. It was found out that clinoptilolite tends to neutralize the solution by acting as H₊ acceptor or H₊ donor (Rivera et al., 2000; Ersoy and celik, 2002). The pH of solution can also affect removal efficiency by affecting the integrity of zeolite. Clinoptilolite is known to partially degrade and lose its ion exchange capacity in alkaline media (Mier et al., 2001). Also, clinoptilolite structure breaks down in highly acidic solutions (Tsitsishvili, 1992). On the other hand, as the solution pH increases, the number of negatively charged sites increases (Benhammou et al., 2005), Clinoptilolite-de-ionized water suspensions at neutral, acidic and basic pH values exhibited a buffer pH around 9±1. This was also observed by Trgo and Peric (2003) and at all initial pH's examined (2-11) in de-ionized water-clinoptilolite suspensions pH became stable between 8 and 9.

Active adsorbent materials such as zeolites, carbon molecular sieve (CMS), alumina and other porous adsorbent materials and lanthanides such as holmium can be coated onto glass fiber paper. In order to bind adsorbent particles with glass fibers and to have uniform distribution of adsorbent particles, many ingredients and additives such as retention binders may also be added into the coating solution. The final non-woven-fabric sheet (paper) will be comprised of the retention aid, the active adsorbent materials and the organic polymer. A retention aid is any material that enhances the retention of the glass fibers in the wall liner and adsorbents.

The retention aid binders such as Alcoa HiQ-40, Alucol or Alumina Sol are added to the slurry to bind the adsorbent particles to the glass fibers in the paper. Through this process, adsorbent particles tend also to be encapsulated by the boehmite binder material.

Absorbent materials such as zeolites adsorbent material which includes but is not limited to zeolite types 5A, 13X, Y, HEU, ZSM-3, EMT, EMC-2, ZSM-18, ZK5, ZSM-5, ZSM-11, .beta., L, chabazite, offretite, mordenite, gmelinite, mazzite, phillipsite and ferrierite and mixtures of these. Other adsorbents such as activated alumina sol, silica gel, carbon molecular sieves and bismuth layered perovskite structured oxides such as SrBi₂Nb₂O₉, SrBi₂Ti₂O₉ and SrBi₂Ta₂O₉ can also be used.

Radiation Absorption Air Filters

Product Description:

High efficiency (HEPA type), high purity, 100% high quality borosilicate glass micro fiber filter media is commonly used in the collection of alpha and beta particles and gamma ray emitting particulates. It is an excellent all around analytical grade filtration media for use in the removal of micron and submicron size particulates from both liquids and gases. HEPA Quality, DOP Collection Efficiency 99.99%, specifically designed for analytical applications (see “Curie Formula”).

Properties of Glass Fiber Media:

The borosilicate glass fibers are inert and resistant to all but strongly alkaline bases or acids such as hydrofluoric acid. The fibers are heat resistant and will only begin to soften at over 500° C. The borosilicate glass has a refractive index of 1.51, and when immersed in a solvent of a similar refractive index like benzene, the fibers will be transparent. Particles collected on the media then become easier to visibly identify.

### Particle Retention:

In nuclear environmental air testing, the protocol is for filtration or collection of sub-micron size (less than one micrometer in aerodynamic diameter) particles. Use of glass fiber media is therefore recommended. The test procedure for determining the effectiveness of particle retention is known as the DOP smoke test. In this test, DOP (dioctyl phthalate) is heated, the vaporized compound is dispersed into the air where it cools and condenses into mono-molecular particles of 0.07-2.0 (˜0.3) micron size. By drawing these airborne particles through the filter media and measuring the amount of breakthrough particle, a retention efficiency is established e.g. 0.001%-0.003% (see Mil Std 282 for the complete procedure).

### SUMMARY OF THE INVENTION

### Objects of the Invention

The present invention generally relates to a method of producing reinforced blended cement (e.g. clinker, synthetic gypsum and petroleum coke powder), plus stainless steel fiber, fly ash and HPC to make high performance concrete for building materials that has increased density, bonding, tensile, flexural and compressive strength.

The present invention also relates to a new application, namely the use of petroleum coke powder and steel fibers as an electrically conductive filler in concrete for electromagnetic interference (EMI) shielding. EMI shielding is in critical demand due to the interference of wireless (particularly radio frequency) devices with digital devices and the increasing sensitivity of electronic devices. Shielding is particularly needed for underground vaults containing transformers and other electronics that are relevant to electric power and telecommunication. It is also needed for deterring electromagnetic forms of spying. The high shielding effectiveness of cement paste containing steel fibers is consistent with its low electrical resistivity. Stainless steel fibers (8 mm diameter) 0.36 vol. % has very low resistivity. The resistivity is 40 Ωcm at 0.78 vol. % steel fibers (8 mm diameter). Hence, steel fibers are effective for passing current. Steel is also much more conductive than carbon. The high conductivity makes steel fibers outstanding for shielding. In spite of the large diameter compared to other shielding materials. In fact, steel fibers (8 mm diameter) at 0.90 vol % reached 71 dB (1.5 GHz). The highest two values of EMI consisted of shielding effectiveness previously reported in cement-matrix composites are 40 dB, as attained in cement paste containing 1.5 vol. % carbon filaments and 70 dB, attained in cement paste containing 0.72 vol. % stainless steel fibers of diameter 8 mm and length 6 mm.

The present invention specifically relates to a new application, namely the use of zeolites (natural (Heu) or synthetic (Ca-A)) and boehmites compounds dissolved in de-ionized water then coated onto a glass fiber substrates (paper) along with an organic wash coated polymer and used to cover HEPA (type) air filtration media and building materials such as wall board and ceiling tiles and panels or as wall liner (covering) for absorption of nuclear fission products such as radioactive isotopes of cesium and strontium and greenhouse gases such as CO2.

The present invention specifically relates to the use of zeolite compound coated radiation absorbing air purifying filters that meet U.S. Military and nuclear specifications and are designed for use in vertical or horizontal (wall mounted) air conditioner units without the need for plasma or electrically conductive components such as manganese dioxide.

Principles in Accordance with the Present Invention

In achievement of the above objects it is suggested that concrete will be reinforced with steel fibers and coal fly ash and the addition of an organic (polysaccharide) admixture e.g. methylcellulose of the invention.

It is also suggested that EMI/RF/Microwave shielding of concrete can be achieved by cross linking or combining cellulose fibers with deflective or absorptive materials such as fly ash containing silica fume (<6 vol. %), coke powder (1.02 vol. %), nickel plated carbon filaments (7 vol. %) or copper coated stainless steel fibers (0.78 vol. %). It is specifically suggested that EMI/RF/Microwave shielded structural and non-structural building materials can be used for lateral and distress guidance systems in automated highways, bridge pavements and levees.

It is also specifically suggested that a moisture based polymer sealant can produce an aqueous environment that will facilitate cation exchanges between trapped cations and negative ions in the cages of the Zeolites.

It is further suggested that positively charged radioactive particles such as ¹³⁷Cesium can be irreversibly trapped in the structural cages within zeolites and the associated electromagnetically positively charged beta stable progeny will be absorbed into the negatively charged magnetic field within the zeolites and the associated gamma rays upon contact with air will generate negative ions that dissipate into the air.

It is further specifically suggested that when the temperature is raised to 300° C. the cages in Phillipsite will collapse around the ¹³⁷Cesium and at temperatures between 800-1000° C. the ¹³⁷Cesium trapped Phillipsite will become stable Pollucite (CsAlSiO+H₂O).

It is also further suggested that the addition of HPC & EMG in de-ionized water will cause an increase in the surface resistivity of zeolite coated fiber glass spun fabric (mat) from 30×10⁷.-824×10⁷ Ohm/Sq adjuvanting EMI absorption.

Lastly, it is suggested that high efficiency (HEPA type), high purity, 100% high quality borosilicate glass micro fiber filter media and wallpaper products produced from the invention can be used in the collection of gamma rays and alpha and beta particle emitting micron and submicron size particulates (PM 0.5-2.5) with DOP collection efficiency >99.99%, which can be specifically designed for absorption of ^{134,137}Cesium and ^{89,90}Strontium and Ozone, Radon and Carbon Dioxide (CO₂) gases. The foregoing discussion discloses and describes merely exemplary embodiments of the present invention. One skilled in the art will readily recognize from such discussion and claims that various changes, modifications and variations can be made therein without departing from the spirit and scope of the invention as defined in the following claims.

### REFERENCES

1. U.S. Pat. No. 6,524,846, U.S. patent application Ser. Nos. 11/110,923, 12/656,741 and 13/067,917 U.S. Provisional Patent Nos. 60/960,071 & 61/006,403, 61/136,183, 61/064,115, 61/136,440, 61/344,491, 61/344,857, 61/457,105, 61/457,155 61/457,156, 61/457,664, 61/457,803 & 61/633,248, Robinson, Jr. William L.
2. Electromagnetic Interference Shielding Effectiveness of Carbon Materials, Composite Materials Research Laboratory, University at Buffalo, State University of New York, Buffalo, N.Y. 14260-4400, USA, D. D. L. Chung, et al, (2001)
3. Cement Based Electromagnetic Shielding and Absorbing Building Materials, Guan, H. et al., Cement and Concrete Research, Vol. (28), Pgs. 468-474. (2006)
4. Organic Chemistry, McMurray J. 5^{th} Edition, Chapter 25, Pgs. 1030-1073 (2000)
5. Journal Of Biochem. Vol. (280) Pgs 530-537 (U.S. Pat. No. 6,906,024)
6. Cereal Chemistry, Vol. (54) Pgs. 266 (1977)
7. Cement and Concrete Research, Pourchez et al, Vol. (36), Pgs. 1252-1256 (2006)
8. Proc. 7^{th} Int. Congr. Chemistry of Cement, Skalny et al, Vol. (I) II-1/32
9. Cement and Concrete Research, Birchall et al, Vol. (13) Pgs. 830-842 (1983)
10. Cement and Concrete Research, Vol. (32), Issue 3, Pgs. 393-399 (March, 2002)
11. (WO/2006/041698) Integrally Waterproofed Concrete, A. Aldykiewicz, Jr. et al. Xypex Chemical Corporation (2008)
12. Use of Fly Ash as an Admixture for Electromagnetic Interference Shielding, Composite Materials Research Laboratory, University at Buffalo, State University of New York, Buffalo, N.Y. 14260-4400, USA, D. D. L. Chung, et al, (2004)
13. Use of Ground Course Fly Ash as a Replacement of Condensed Silica Fume in Producing High Strength Concrete, Chai, J. et al., Department of Civil Engineering, King Mongkut's University of Technology, Thonburi, Bankok 10140, Thailand (2003)
14. Agent for trapping the radioactivity of fission products which are generated in a nuclear fuel element, Marsaud, et al., U.S. Pat. No. 5,272,740, (1993)
15. Rivera A., Rodriguez-Fuentes G., Altshuler E. (2000). Time evolution of a natural clinoptilolite in aqueous medium: conductivity and pH experiments. Microporous and Mesoporous Materials, 40, 173-179.
16. Trgo M., Peric J. (2003) Interaction of the zeolitic tuff with Zn-containing simulated pollutant solutions. Journal of Colloid and Interface Science, 260, 166-175.
   a. Phillipsite In Cs Decontamination And Immobilization, Sridhar Komarneni. Clays and Clay Minerals, Vol. 33, No. 2, 145-151, 1985.
   a. Titanium Dioxide for Environmental Catalysis, (2013) Cristal Global
18. Corporation,www.cristal.com

What is claimed is:

1. A Method of producing wall coverings and air filtration media containing absorbent materials including Clinoptilolite (HEU) or Ca-A, Type 5A (Zeolite) as the trapping agent dissolved in de-ionized water along with a retention aid and a sealant polymer (Curie Formula) coated onto woven or nonwoven glass fibers or cellulose based paper and/or polypropylene reinforced spunbond media for the purposes of providing EMI and radioisotope shielding of building materials, for absorption of airborne particulates such as PM 0.5-2.5 and for absorbing CO₂ for climate change mitigation comprising:
a) the step of mixing radiation absorbing materials˜60-80%-325 mesh zeolites and correspondingly 40-20% boehmite (retention aid) binder in de-ionized water (5:1 ratio) at pH 8-9, specifically 8.5-8.9 at 28-30° C., specifically 28.8° C., and an organic polymer containing Hydroxypropylcellulose (HPC)+Ethoxylated Methylglucoside (EMG)˜60%:40% (ratio) (to minimize particle separation from the substrate creating dust), for two (2) minutes, then,
b) the step of applying (spraying or dipping) or coating the absorbing material onto one side or both sides of a [cellulose paper, glass fiber paper or polypropylene reinforced spunbond meltblown air filtration media and aluminum foil backed paper] substrate,
then,
c) the step of applying (coating) an organic polymer over the radiation absorbing coated material (glass fiber paper, borosilicate glass fiber paper, cellulose paper or polypropylene reinforced spunbond air filtration media or cellulose paper) containing Hydroxypropylcellulose (HPC)+(EMG)˜60%:40% (ratio) in de-ionized water (20% vol. wt) to adjuvant EMI attenuation by increasing the surface resistivity from 30×10⁷ Ohms/Sq up to 824×10⁷ Ohms/Sq. Substrates including ULPA and HEPA type (<0.3 microns) air filtration media may be coated with a Mayer Rod. The bottom (first layer) dry aim thickness is 1.8 mil and the top or (sealant layer) is 1.4 mil, air penetration is 0.001%-0.003%
and
d) the step of binding commercial grade spun woven or nonwoven products such as Typal® (produced by the Fiberweb Corporation) to the underside of borosilicate air filtration media to reinforce its strength to support increased airflow through the media by stitching or using chemical adhesives.
or
e) the step of mixing 20% 5A Zeolite and 5% Boehmite with 25% HPC+EMG and 50% Deionized water, pH 8.5 @ at room temperature and then coating this mixture onto one side or both sides of a glass fiber borosilicate or polypropylene substrate e.g., air filtration media (primary surface) then
f) the step of mixing 33% HPC+EMG and 33% TiO₂ to 33% Deionized water and then
recoating the primary coated layer for permanent sealing. Substrates including ULPA or HEPA Type (<0.3 microns) polypropylene air filtration media may be coated with a Mayer rod. The bottom (first layer) dry aim thickness is 1.8 mil and the top (or sealant) layer is 1.4 mil, air penetration is 0.001%-0.003%. for borosilicates and 0.31%-0.89% for polypropylene (see Table—Tests Results) and
g) the step of laminating a (0.001″-0.002″) layer of aluminum or copper foil onto the underside of zeolite coated paper for radiation reflection,
or
h) the step of mixing 20% HEU Zeolite and 5% Boehmite with 25% HPC+EMG and 50% Deionized water, pH 8.5 @ at room temperature and then coating mixture onto one side or both sides of a glass fiber borosilicate or polypropylene substrate e.g., air filtration media (primary surface) then
i) the step of mixing 33% HPC+EMG and 33% SrBi₂Ti₂O₉ (or TiO₂) to 33% Deionized water and then
recoating the primary coated layer for permanent sealing. Substrates including ULPA or HEPA Type (<0.3 microns) polypropylene air filtration media

2. The use of a moisture laden polymer sealant according to claim 1 composed of HPC+EMG+H₂O to produce an aqueous environment for ion (cation) exchange to occur.

3. The use of coated glass fiber, cellulose or borosilicate paper according claim 1 wherein cation particles remain trapped in the crystalline structure of zeolites until they permanently decay to stable elements such as Barium or Yttrium and are recycled commercially.

4. Absorbent materials according to claim 1, such as zeolite adsorbent materials includes but are not limited to zeolite type X, zeolite type 5A, zeolite type Y, ZSM-3, EMT, EMC-2, ZSM-18, ZK5, ZSM-5, ZSM-11, .TS-1 (titanium silicates) titanium dioxide, beta., L, chabazite, offretite, erionite, mordenite, gmelinite, mazzite, phillipsite, brewsterite and mixtures of these. Other adsorbents such as activated alumina sol, silica gel, carbon molecular sieves, amorphous aluminosilicate, clay materials and bismuth layered perovskite structured oxides such as SrBi2Nb2O0, SrBiTio9 and SrbiTaO9 can also be used.

5. The use of desorbed (previously trapped in air filtration media) Cesium according to claim 1, for the immobilization of Uranium and its alpha and beta particles and other transuranic metals including Polonium and Plutonium.

6. The use of ⁷Be as a marker for submicron size airborne radioactive particles (<1.0 μm) absorption into coated air filtration media according to claim 1.

7. The use of clinically therapeutic products produced according to claim 1, from desorbed Cesium combined with chloride.

8. The use of desorbed Cesium according to claim 1, as a source of ions for use as an intergalactic rocket fuel propellent instead of gaseous combustion or the use of other desorbed transuranic elements such as Plutonium.

9. A radioactivity trapping agent containing a negatively charged stable fissionable product absorbing crystalline structure and at least one metallic or paramagnetic oxide of non-radioactive substance such as titanium dioxide which is used with a retention aid as a photocatalyst for filtration of airborne viruses, bacteria, mold and mildew from indoor air.

10. The retention aid binders according to claim 9, such as BASF (Alcoa) HiQ-40, or Alumina Sol are added to the slurry to bind the adsorbent particles to the glass or cellulose fibers in the paper. Through this process, adsorbent particles tend also to be encapsulated by the boehmite binder material.

11. A trapping agent according to claim 9, wherein in the stable oxygenated compound and the metallic oxides are selected from the group consisting of Al₂O₃, SiO₂, and wherein a zeolites e.g. Phillipsite containing Cesium is heated to 300-500° C. and the internal cages surrounding the trapped radioactive particle starts to collapse and when heated to 800-1000° C. the resultant stable material produced is Pollucite which is used to capture other beta decay particles (positrons) and [absorption of] gamma radiation from ¹³⁷B.

12. A radiation absorbing air purifying filter according to claim 9 that meets U.S. Military Standard 282 DOP and international nuclear specifications and is designed for use with vertical or horizontal wall mounted (inverted) air conditioner units with or without the use of plasma or electrical conductive photocatalytic components such as titanium dioxide or titanium silicate that attaches directly to removable filter frames.

13. The use of absorbed ozone according to claim 12 to deactivate or decompose airborne bacteria, viruses, mold and mildew.

14. The use of aluminum silicate compounds (zeolites) according to claim 12 to trap radon gas and retain its daughter progeny such as polonium which causes lung and breast cancer.

15. The use of aluminum silicate compounds (zeolites) according to claim 12 to trap and absorb EMI radiation (waves) for cybersecurity.

16. The use of bismuth layered perovskite structured oxides such as SrBi₂Ti₂O₉ or ferrierite coated aluminum silicate compounds (zeolites) according to claim 12 to trap and retain CO₂, CH₄, H₂S and NOx which causes global warming.

17. The use of Ca-A 5(A) synthetic zeolite coated media according to claim 12 for absorption of CO₂ in flue gases produced from the production and combustion of fossil fuels such as coal, petroleum and natural gas.
